(19) United States
(12) Patent Application Publication
(10) Pub. No.: US 2021/0282721 A1
(43) Pub. Date: Sep. 16, 2021

(54) Biological Information Analysis Device, Biological Information Analysis Method, and Biological Information Analysis System

(71) Applicant: Nippon Telegraph and Telephone Corporation, Tokyo (JP)

(72) Inventors: Takayuki Ogasawara, Tokyo (JP); Rieko Sato, Tokyo (JP)

(21) Appl. No.: 17/271,286

(22) PCT Filed: Aug. 22, 2019
(86) PCT No.: PCT/JP2019/032788

§ 371 Date: Feb. 25, 2021

(30) Foreign Application Priority Data
Sep. 5, 2018 (JP) 2018-165811

(51) Int. Cl. A61B 5/00; G16H 50/30; G16H 40/67; G16H 15/00; A61B 5/024

(52) U.S. Cl. CPC A61B 5/7282; G16H 50/30; A61B 5/332; G16H 15/00; A61B 5/02438; G16H 40/67

(57) ABSTRACT

A biological information analysis device includes a sensor data acquisition unit that acquires the biological information measured by a sensor and a data analysis unit that analyzes time-series data of the biological information over a plurality of times periods to calculate, from a plurality of biological information sets acquired at the mutually corresponding measurement times in the plurality of individual time periods, a representative value of the plurality of biological information sets.

### CROSS-REFERENCE TO RELATED APPLICATIONS

This application is a national phase entry of PCT Application No. PCT/JP2019/032788, filed on Aug. 22, 2019, which claims priority to Japanese Application No. 2018-165811, filed on Sep. 5, 2018, which applications are hereby incorporated herein by reference.

### TECHNICAL FIELD

The present invention relates to a biological information analysis device, a biological information analysis method, and a biological information analysis system, and particularly to a technique for analyzing biological information measured by a sensor worn by a user BACKGROUND

In recent years, in sports and medicine, biological information such as a heart rate and an amount of activity is measured using a wearable device or the like. For example, NPL 1 discloses a technique for stably measuring, over a long period of time, a heart rate or an electrocardiographic waveform of a user wearing a wearable bioelectrode inner made of a fibrous conductive textile material. NPL 1 also discloses a technique for estimating a posture or a gait of the user on the basis of measurement data from an acceleration sensor embedded in the wearable device worn by the user.

### CITATION LIST

### Non Patent Literature

NPL 1—Kasai, Ogasawara, Nakashima, and Tsukata “Development of Functional Textile “hitoe”: Wearable Electrodes for Monitoring Human Vital Signals”, The Institute of Electronics, Information and Communication Engineers IEICE Communications Society Magazine Issue No. 41 (June 2017) (Vol. 11 No. 1).

### SUMMARY

### Technical Problem

The conventional techniques allow the biological information of the user to be continuously and stably measured. However, the user do not perform the same routine activities every day. Accordingly, when the routine activities performed by the user vary from one day to another, it is difficult to obtain a typical value of the biological information at respective measurement times in time periods such as one day unit period or one hour unit period.

Embodiments of the present invention are achieved in view of the problem described above, and an object of embodiments of the present invention is to provide a biological information analysis device, a biological information analysis method, and a biological information analysis system which allow a typical value of biological information at respective measurement times in set time periods to be obtained.

### Means for Solving the Problem

To solve the problem described above, a biological information analysis device according to the present embodiment includes: a sensor data acquisition unit that acquires biological information measured by a sensor; and a data analysis unit that analyzes time-series data of the biological information over a plurality of time periods to calculate, from a plurality of biological information sets acquired at mutually corresponding measurement times in the plurality of individual time periods, a representative value of the plurality of biological information sets.

The biological information analysis device according to embodiments of the present invention further includes: an abnormal value determination unit that determines, on the basis of a preset reference, whether or not the plurality of biological information sets include an abnormal value, wherein, when it is determined by the abnormal value determination unit that the abnormal value is included, the data analysis unit may also calculate, from the plurality of biological information sets acquired at the mutually corresponding measurement times in the plurality of individual time periods from which the abnormal value is excluded, the representative value of the plurality of biological information sets.

In the biological information analysis device according to embodiments of the present invention, the data analysis unit may also include: an averaging processing unit that calculates, as the representative data, an average value of the plurality of biological information sets.

In the biological information analysis device according to embodiments of the present invention, the data analysis unit further includes: a summarization unit that calculates, on the basis of time-series data of the average value of the plurality of biological information sets, a summary value which is a value obtained by statistically summarizing the plurality of biological information sets for each of randomly selected periods included in the plurality of time periods, and the summary value may also include at least one of a cumulative total value, an average value, a detailed breakdown representing a proportion accounted for by a total duration period of a randomly selected value of the biological information in each of the time periods, a median value, a 25% level point, a 75% level point, a standard deviation, and a standard error.

In the biological information analysis device according to embodiments of the present invention, the summarization unit may also calculate biological state information, which is a qualitative variable, on the basis of the time-series data of the biological information over the plurality of time periods, and calculate the summary value by statistically summarizing the biological state information on the basis of frequencies of a value of the biological state information at mutually corresponding measurement times in the plurality of individual time periods.

To solve the problem described above, a biological information analysis method according to the present embodiment includes: a sensor data acquisition step of acquiring biological information measured by a sensor; and a data analysis step of analyzing time-series data of the biological information over a plurality of time periods to calculate, from a plurality of biological information sets acquired at mutually corresponding measurement times in the plurality of individual time periods, a representative value of the plurality of biological information sets.

To solve the problem described above, a biological information analysis system according to the present embodiment includes: a sensor terminal that outputs, to an outside thereof, biological information measured by a sensor worn by a user; a relay terminal that receives the biological information output from the sensor terminal and outputs, to an outside thereof, the biological information; and an external terminal that receives the biological information output from the sensor terminal or the relay terminal and causes a display device to display the biological information, wherein at least any of the sensor terminal, the relay terminal, and the external terminal includes: a sensor data acquisition unit that acquires the biological information; a data analysis unit that analyzes time-series data of the biological information over a plurality of time periods to calculate, from a plurality of biological information sets acquired at mutually corresponding measurement times in the plurality of individual time periods, a representative value of the plurality of biological information sets; and a presentation unit that outputs the representative value of the plurality of biological information sets as a portion of the biological information.

A biological information analysis system according to embodiments of the present invention includes a sensor terminal having a first data analysis unit; a relay terminal having a second data analysis unit; and an external terminal having a third data analysis unit, wherein the sensor terminal outputs, to an outside thereof, biological information measured by a sensor worn by a user, the relay terminal receives the biological information output from the sensor terminal and outputs, to an outside thereof, the biological information, the external terminal receives the biological information output from the sensor terminal or the relay terminal and causes a display device to display the biological information, and the first data analysis unit, the second data analysis unit, and the third data analysis unit cooperate to analyze time-series data of the biological information over a plurality of time periods to calculate, from a plurality of biological information sets acquired at mutually corresponding measurement times in the plurality of individual time periods, a representative value of the plurality of biological information sets.

### Effects of Embodiments of the Invention

According to embodiments of the present invention, the time-series data of the biological information over the plurality of time periods is analyzed and, from the plurality of biological information sets acquired at the mutually corresponding measurement times in the plurality of individual time periods, the representative value of the plurality of biological information sets is calculated. Therefore, it is possible to obtain the typical value of the biological information at the respective measurement times in the set time periods.

### BRIEF DESCRIPTION OF THE DRAWINGS

FIG. 1 is a block diagram illustrating functions of a biological information analysis device according to a first embodiment of the present invention.

FIG. 2 is a block diagram illustrating a hardware configuration of the biological information analysis device according to the first embodiment.

FIG. 3 is a flow chart illustrating a biological information analysis method according to the first embodiment.

FIG. 4 is a graph illustrating processing of averaging heart rates according to the first embodiment.

FIG. 5 is a diagram illustrating a configuration of a biological information analysis system according to the first embodiment.

FIG. 6 is a block diagram illustrating a configuration of the biological information analysis system according to the first embodiment.

FIG. 7 is a sequence diagram illustrating an operation of the biological information analysis system according to the first embodiment.

FIG. 8 is a block diagram illustrating functions of a biological information analysis device according to a second embodiment.

FIG. 9 is a sequence diagram illustrating an operation of a biological information analysis system according to the second embodiment.

FIG. 10 is a graph illustrating processing of determining an abnormal value of a heart rate according to the second embodiment.

FIG. 11 is a block diagram illustrating functions of a biological information analysis device according to a third embodiment.

FIG. 12 is a sequence diagram illustrating an operation of a biological information analysis system according to the third embodiment.

FIG. 13 is a block diagram illustrating functions of a biological information analysis device according to a fourth embodiment.

FIG. 14 is a sequence diagram illustrating an operation of a biological information analysis system according to the fourth embodiment.

FIG. 15 is a graph illustrating calculation of a summary value according to the fourth embodiment.

### DETAILED DESCRIPTION OF ILLUSTRATIVE EMBODIMENTS

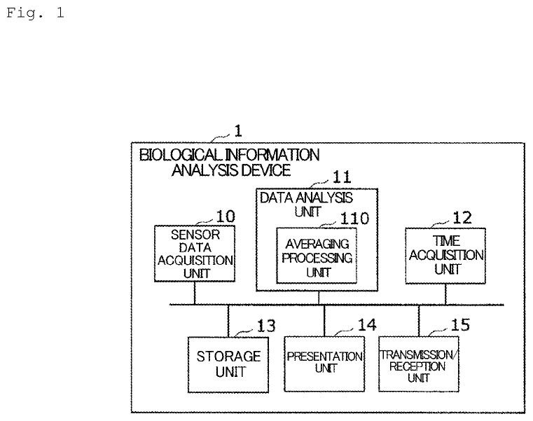

Referring to FIGS. 1 to 15, a detailed description will be given of preferred embodiments of the present invention.

### First Embodiment

First, a description will be given of an outline of a configuration of a biological information analysis device 1 according to the first embodiment of the present invention. FIG. 1 is a block diagram illustrating a functional configuration of the biological information analysis device 1.

Functional Block of Biological Information Analysis Device

The biological information analysis device 1 includes a sensor data acquisition unit 10, a data analysis unit 11, a time acquisition unit 12, a storage unit 13, a presentation unit 14, and a transmission/reception unit 15.

The sensor data acquisition unit 10 acquires, from a sensor 106 worn by a user and described above, biological information of the user measured by the sensor 106. More specifically, the sensor data acquisition unit 10 calculates a heart rate represented by digital data from an electrocardiographic waveform based on, e.g., a cardiac potential measured by a heart rate measurement device. The sensor data acquisition unit 10 also converts an analog acceleration signal measured by an acceleration sensor to a digital signal at a predetermined sampling rate. The sensor data acquisition unit 10 outputs time-series data in which the heart rate or the acceleration signal represented digital data is associated with measurement times. The time-series data of the biological information acquired by the sensor data acquisition unit 10 is stored in the storage unit 13 described later.

The data analysis unit 11 includes an averaging processing unit 110. The data analysis unit 11 analyzes the time-series data of the biological information of the user acquired by the sensor data acquisition unit 10 to calculate, from a plurality of biological information sets acquired at mutually corresponding measurement times in a plurality of individual time periods, a representative value representing a typical value of the plurality of biological information sets. Note that, as each of the time periods, any time length such as, e.g., one minute, one hour, one day, one month, or one year can be set.

The averaging processing unit 110 calculates, on the basis of the time-series data of the biological information acquired by the sensor data acquisition unit 10, an average value of the plurality of biological information sets acquired at the mutually corresponding measurement times in the plurality of individual time periods as the representative value. In one example, when it is assumed that each of the time periods is one day, the averaging processing unit 110 calculates, from the time-series data of the biological information over a plurality of days, the average value of the plurality of biological information sets measured at the same time.

In another example, when it is assumed that each of the time periods is one hour, the averaging processing unit 110 may appropriately calculate the average value of the plurality of biological information sets measured at the same time in minutes from the time-series data of the biological information over a plurality of hours. In still another example, when it is assumed that each of the time periods is one minute, the averaging processing unit 110 may appropriately calculate the average value of the plurality of biological information sets measured at the same time in seconds from the time-series data of the biological information over a plurality of minutes.

Note that the plurality of biological information sets acquired at the mutually corresponding measurement times the average value of which is to be calculated by the averaging processing unit 110 need not be biological information sets sampled strictly at the same time, and may appropriately be biological information sets measured in a time range which can be considered as a range near the time.

The time acquisition unit 12 acquires a reference time to be used in the biological information analysis device 1. The time acquisition unit 12 may also acquire time information from, e.g., a built-in clock provided in the biological information analysis device 1 or a time server. The time information acquired by the time acquisition unit 12 is used for data analysis such as averaging processing of the biological information in the data analysis unit 11.

The storage unit 13 stores time-series data of the biological information of the user acquired by the sensor data acquisition unit 10. The storage unit 13 also stores set information related to time periods and a result of the analysis of the biological information by the data analysis unit 11.

The presentation unit 14 presents the result of the analysis by the data analysis unit 11. More specifically, the presentation unit 14 displays the analysis result on a display device 109 described later or generates information supporting the user and presents the user support information on the basis of the analysis result. The presentation unit 14 may also output the information supporting the user to an operating device (not shown) implemented by the display device 109, an audio output device, a light source, an actuator, a heating device, or the like.

The transmission/reception unit 15 receives sensor data representing the biological information measured by the sensor 106 described later. The transmission/reception unit 15 can transmit, to an outside thereof, the result of the analysis of the biological information by the data analysis unit 11 via a communication network.

The individual functions of the biological information analysis device 1 described above may be not only provided in one computer, but also configured to be distributed to a plurality of calculators communicatively connected to each other via the communication network.

Hardware Configuration of Biological Information Analysis Device

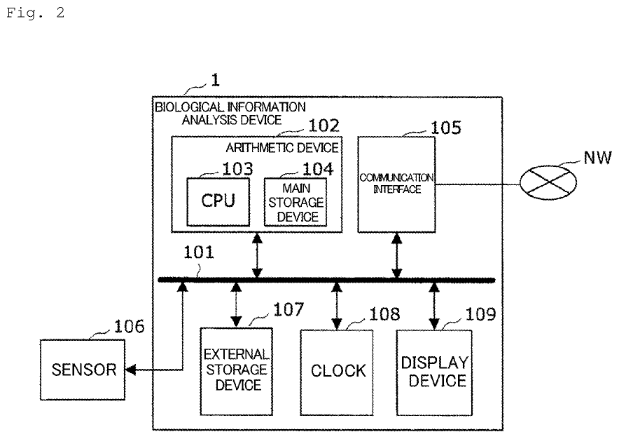

Next, using a block diagram in FIG. 2, a description will be given of an example of a hardware configuration of the biological information analysis device 1 having the functions described above.

As illustrated in FIG. 2, the biological information analysis device 1 can be implemented by, e.g., a computer including an arithmetic device 102 including a CPU 103 and a main storage device 104 which are connected via a bus 101, a communication interface 105, the sensor 106, an external storage device 107, a clock 108, and the display device 109 and by programs for controlling such hardware resources.

The CPU 103 and the main storage device 104 are included in the arithmetic device 102. In the main storage device 104, programs for allowing the CPU 103 to perform various control and arithmetic operations are stored in advance. The arithmetic device 102 implements the individual functions of the biological information analysis device 1 including the data analysis unit 11 illustrated in FIG. 1.

The communication interface 105 is an interface and a control device each for providing communication between the biological information analysis device 1 and various external electronic devices via a communication network NW. The biological information analysis device 1 may also receive, via the communication network NW, data on a heart rate, an electrocardiographic waveform, and an acceleration from the sensor 106 attached to the user via the communication interface 105 and described later.

As the communication interface 105, an arithmetic interface and an antenna which are compliant with a wireless data communication standard such as, e.g., LTE, 3G, a wireless LAN, or Bluetooth (registered trademark). The communication interface 105 implements the transmission/reception unit 15 illustrated in FIG. 1.

The sensor 106 is implemented by a sensor such as, e.g., a heart rate measurement device, an electrocardiograph, or an acceleration sensor. The sensor 106 is worn by the user over a preset measurement period to measure biological information such as a heart rate, an electrocardiographic waveform, or an acceleration of the user.

The external storage device 107 is configured to include a readable/writable storage medium and a drive device for reading/writing various information such as programs or data to the storage medium. For the external storage device 107, a semiconductor memory such as a hard disk or a flash memory can be used as the storage medium.

The external storage device 107 includes a storage region in which time-series data of the biological information measured by the sensor 106 is to be stored, a program storage unit which stores programs for allowing the biological information analysis device 1 to perform processing of analyzing the biological information, and another storage device not shown. For example, the external storage device 107 can include a storage device for backing up the programs, the data, and the like which are stored in the external storage device 107 or the like. The external storage device 107 implements the storage unit 13 illustrated in FIG. 1.

The clock 108 is formed of the built-in clock provided in the biological information analysis device 1 to perform time measurement. Time information obtained by the clock 108 is used for sampling of the biological information and data analysis processing. Note that the time information obtained by the clock 108 is acquired by the time acquisition unit 12 illustrated in FIG. 1.

The display device 109 functions as the presentation unit 14 of the biological information analysis device 1. The display device 109 is implemented by a liquid crystal display or the like. The display device 109 is also included in the operating device that outputs the user support information generated on the basis of the result of analyzing the biological information.

Biological Information Analysis Method

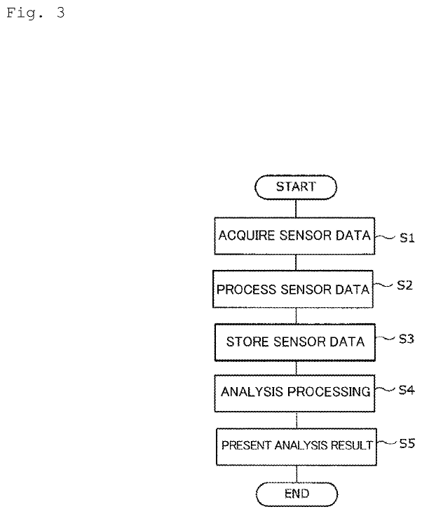

Next, using a flow chart in FIG. 3, a description will be given of an operation of the biological information analysis device 1 having the configuration described above. First, the following processing is performed with the sensor 106 being worn by the user.

The sensor data acquisition unit 10 acquires, via the transmission/reception unit 15, the biological information measured by the sensor 106 worn by the user (Step S1). More specifically, when, e.g., “one day” is set as each of the time periods, the sensor data acquisition unit 10 acquires the electrocardiographic waveform of the user measured over the plurality of time periods such as, e.g., two days. The acquired biological information over the two days is stored in the storage unit 13. Then, the sensor data acquisition unit 10 performs removal of noise from the acquired biological information, and also performs processing of converting the biological information represented by an analog signal to a digital signal (Step S2).

Specifically, the sensor data acquisition unit 10 performs noise removal through filtering from a cardiac potential measured by the heat rate measurement device, and also calculates a heart rate represented by digital data from the electrocardiographic waveform based on the cardiac potential. The time-series data of the biological information processed by the sensor data acquisition unit 10 is stored in the storage unit 13 (Step S3).

Next, the data analysis unit 11 performs processing of analyzing the biological information acquired in Step S2 (Step S4). More specifically, the averaging processing unit 110 calculates an average value of respective heart rates acquired at mutually corresponding measurement times on the first and second days from the time-series data of the heart rates over the two days (two time periods).

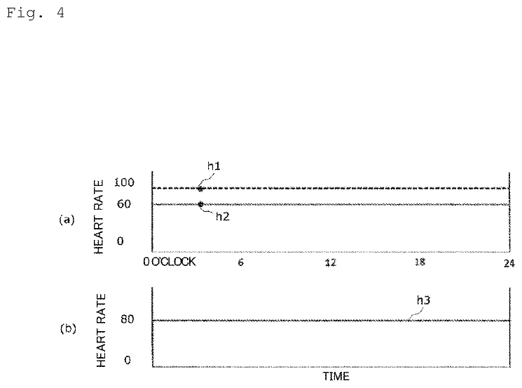

(a) of FIG. 4 is a graph illustrating values of the heart rates measured over the two days. An abscissa axis in (a) of FIG. 4 represents time, while an ordinate axis therein represents the heart rate. A reference numeral h1 denotes the heart rate on the first day (during the first time period). A reference numeral h2 denotes the heart rate on the second day (during the second time period). As illustrated in (a) of FIG. 4, it is simulated that the heart rate remained unchanged at 100 bpm on the first day and remained unchanged at 60 bpm on the second day. The average value of these heart rates is given by Expression (1) below.

$$\begin{matrix}Formula 1 & \\ {A}_{i} = \frac{{\sum }_{i = 1}^{N} {X}_{t , i}}{N} & ( 1 )\end{matrix}$$

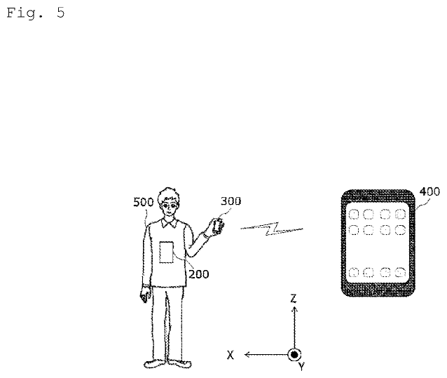

In Expression (1) above, t represents a measurement time, and measurement is performed at a measurement frequency based on a sampling rate, while N represents the number of days over which measurement was performed, i.e., the time periods. N also represents the number of data sets at the mutually corresponding measurement times in the plurality of individual time periods. In the example of FIG. 5, N=2 is satisfied. In Expression (1), i denotes a measurement day (time period) and corresponds to each of the measurement days. In the case of (a) of FIG. 4, an average value At is constantly 80 bpm ((100+60)/2=80). Accordingly, as illustrated in (b) of FIG. 4, the average value of the heart rates at the mutually corresponding measurement times in the two days is 80 bpm (reference numeral h3).

By thus calculating the average value of the plurality of biological information sets acquired at the mutually corresponding measurement times in the plurality of individual days, it is possible to reduce day-by-day fluctuations in the heart rate of the user measured over the plurality of days (in the plurality of time periods) and obtain a measurement value of the biological information close to a real habit or behavior of the user.

Back to the flow chart in FIG. 3, a result of analyzing the biological information of the user obtained by the data analysis unit 11 is stored in the storage unit 13. In addition, the presentation unit 14 displays the analysis result on the display device 109 (Step S5). The presentation unit 14 also generates the support information for the user on the basis of the analysis result and displays the support information on the display device 109 or the like.

Biological Information Analysis System

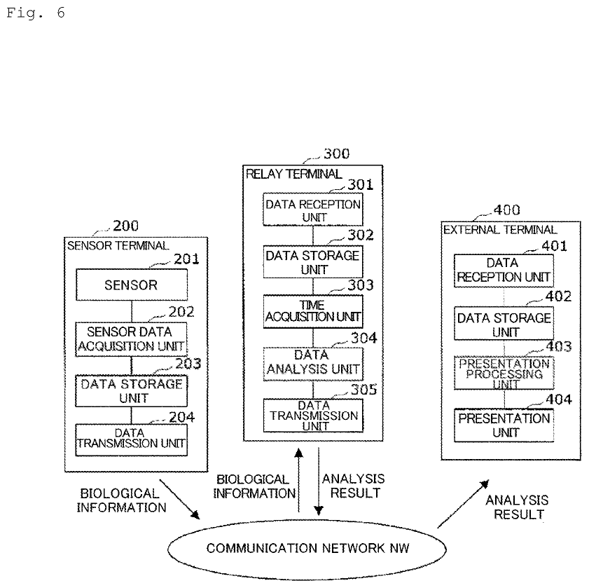

Next, referring to FIGS. 5 and 6, a description will be given of a biological information analysis system obtained by specifically configuring the biological information analysis device 1 according to embodiments of the present invention.

As illustrated in, e.g., FIG. 5, the biological information analysis system includes a sensor terminal 200 to be worn by a user 500, a relay terminal 300, and an external terminal 400. All or any of the sensor terminal 200, the relay terminal 300, and the external terminal 400 has any of the functions of the biological information analysis device 1 such as the function of the data analysis unit 11 illustrated in FIG. 1. Note that the following will describe a case where the relay terminal 300 includes the data analysis unit 11 illustrated in FIG. 1.

Functional Block of Sensor Terminal

The sensor terminal 200 includes a sensor 201, a sensor data acquisition unit 202, a data storage unit 203, and a data transmission unit 204. For example, the sensor terminal 200 is placed on a body trunk of a body of the user 500 to measure biological information over a plurality of time periods. The sensor terminal 200 transmits the measured biological information of the user 500 to the relay terminal 300 via the communication network NW.

The sensor 201 is implemented by a heart rate measurement device, an acceleration sensor, or the like. Three axes of the acceleration sensor included in the sensor 201 are provided such that, e.g., an X-axis is parallel with a left-right direction of the body, a Y-axis is parallel with a front-rear direction of the body, and a Z-axis is parallel with a vertical direction of the body as illustrated in FIG. 5. The sensor 201 corresponds to the sensor 106 illustrated in FIG. 2.

The sensor data acquisition unit 202 acquires the biological information measured by the sensor 201. More specifically, the sensor data acquisition unit 202 performs noise removal and sampling processing on the acquired biological information to obtain time-series data of the biological information represented by a digital signal. The sensor data acquisition unit 202 corresponds to the sensor data acquisition unit 10 illustrated in FIG. 2.

The data storage unit 203 stores the time-series data of the biological information measured by the sensor 201 and the time-series data of the biological information represented by the digital signal resulting from the processing by the sensor data acquisition unit 202. The data storage unit 203 corresponds to the storage unit 13 (FIG. 1).

The data transmission unit 204 transmits the time-series data of the biological information stored in the data storage unit 203 to the relay terminal 300 via the communication network NW. For example, the data transmission unit 204 includes a communication circuit for performing wireless communication compliant with a wireless data communication standard such as, e.g., LTE, 3G, a wireless LAN (Local Area Network), or Bluetooth (registered trademark). The data transmission unit 204 corresponds to the transmission/reception unit 15 (FIG. 1).

Functional Block of Relay Terminal

The relay terminal 300 includes a data reception unit 301, a data storage unit 302, a time acquisition unit 303, a data analysis unit 304, and a data transmission unit 305. The relay terminal 300 analyzes the time-series data of the biological information of the user 500 measured over the plurality of time periods and received from the sensor terminal 200. In addition, the relay terminal 300 calculates, from a plurality of biological information sets acquired at mutually corresponding measurement times in the plurality of time periods, a representative value of the plurality of biological information sets and transmits the representative value as a result of analysis to the external terminal 400.

The relay terminal 300 is implemented by a smart phone, a tablet, a notebook personal computer, or the like.

The data reception unit 301 receives the time-series data of the biological information from the sensor terminal 200 via the communication network NW. The data reception unit 301 corresponds to the transmission/reception unit 15 (FIG. 1).

The data storage unit 302 stores the biological information of the user 500 received by the data reception unit 301 and the result of the analysis of the biological information by the data analysis unit 304. The data storage unit 302 corresponds to the storage unit 13 (FIG. 1).

The time acquisition unit 303 acquires, from the built-in clock (not shown), time information to be used in the processing of analyzing the biological information performed by the data analysis unit 304. The time acquisition unit 303 corresponds to the time acquisition unit 12 illustrated in FIG. 1.

The data analysis unit 304 analyzes the time-series data of the biological information of the user 500 over the plurality of time periods that has been received by the data reception unit 301. Then, the data analysis unit 304 obtains, from the plurality of biological information sets acquired at the mutually corresponding measurement times in the plurality of time periods, the representative value representing a typical value of the plurality of biological information sets. For example, the data analysis unit 304 calculates an average value of heart rates of the user 500 measured in the same time block over a plurality of days using Expression (1) described above. The average value of the biological information sets obtained as a result of analysis is stored in the data storage unit 302. The data analysis unit 304 corresponds to the data analysis unit 11 including the averaging processing unit 110 illustrated in FIG. 1.

The data transmission unit 305 transmits the result of the analysis by the data analysis unit 304 to the external terminal 400 via the communication network NW. The data transmission unit 305 corresponds to the transmission/reception unit 15 (FIG. 1).

Functional Block of External Terminal

The external terminal 400 includes a data reception unit 401, a data storage unit 402, a presentation processing unit 403, and a presentation unit 404. The external terminal 400 presents the result of the analysis of the biological information of the user 500 received from the relay terminal 300 via the network NW and presents the support information for the user 500 based on the analysis result.

Similarly to the relay terminal 300, the external terminal 400 is implemented by a smart phone, a tablet, a notebook personal computer, or the like. The external terminal 400 includes a display device that displays the received analysis result and an operating device (not shown) that outputs the information supporting the user 500 generated on the basis of the analysis result. Examples of the operating device included in the external terminal 400 include a display device, an audio output device, a light source, an actuator, a heating device, and the like.

As an example of the audio output device, a speaker or a musical instrument may also be used. As the light source, an LED or an electric bulb may also be used. As the actuator, an oscillator, a robot arm, or an electrical therapeutic device may also be used. As the heating device, a heater, a Peltier element, or the like may also be used.

The data reception unit 401 receives the result of the analysis of the biological information from the relay terminal 300 via the communication network NW. The data reception unit 401 corresponds to the transmission/reception unit 15 (FIG. 2).

The data storage unit 402 stores the result of the analysis of the biological information received by the data reception unit 401. The data storage unit 402 corresponds to the storage unit 13 (FIG. 1).

The presentation processing unit 403 generates the support information for the user 500 on the basis of the analysis result. The presentation processing unit 403 corresponds to the presentation unit 14 illustrated in FIG. 1.

The presentation unit 404 presents the support information for the user 500 on the basis of the presentation of the analysis result or an instruction from the presentation processing unit 403. More specifically, the presentation unit 404 may also display the analysis result or the support information on a display device included in the external terminal 400 by using textural information, a graph, or the like or output the support information by using an alert from a speaker included in the external terminal 400 and not shown or the like. Besides, the presentation unit 404 can present the support information using a method recognizable by the user 500, such as vibration or light. The presentation unit 404 corresponds to the presentation unit 14 illustrated in FIG. 1.

Thus, the biological information analysis system according to embodiments of the present invention has a configuration in which the individual functions of the biological information analysis device 1 are distributed to the sensor terminal 200, the relay terminal 300, and the external terminal 400, and performs processing related to the acquisition of the biological information by the user 500, the analysis of the biological information, and the presentation of the analysis result in a distributed manner.

Operation Sequence of Biological Information Analysis System

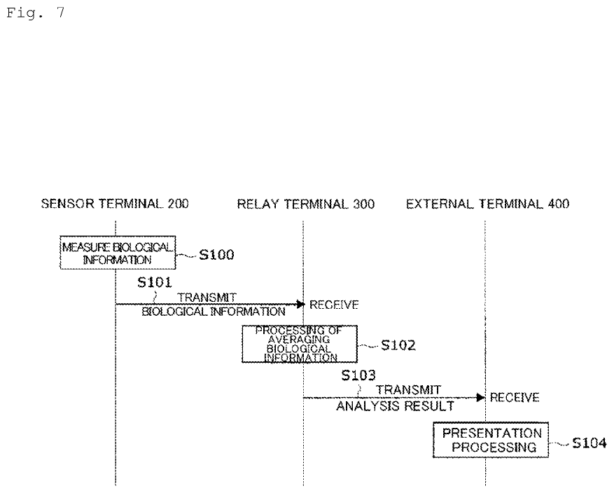

Next, using a sequence diagram in FIG. 7, a description will be given of an operation of the biological information analysis system having the configuration described above.

As illustrated in FIG. 7, first, the sensor terminal 200 is worn by the user 500 to measure biological information over a plurality of time periods (Step S100). The sensor terminal 200 obtains a digital signal representing the measured biological information, and removes noise therefrom as required.

Then, the sensor terminal 200 transmits the biological information to the relay terminal 300 via the communication network NW (Step S101). When receiving time-series data of the biological information from the sensor terminal 200, the relay terminal 300 performs averaging processing to perform analysis of the biological information (Step S102). More specifically, the data analysis unit 304 (averaging processing unit 110) of the relay terminal 300 calculates, from a plurality of biological information sets acquired at mutually corresponding measurement times in the plurality of individual time periods, an average value of the plurality of biological information sets by using Expression (1) described above.

Then, the relay terminal 300 transmits, to the external terminal 400, a result of the analysis representing the average value of the biological information sets via the communication network NW (Step S103). When receiving the analysis result, the external terminal 400 performs presentation processing (Step S104). Specifically, the external terminal 400 displays the analysis result on the display device. Alternatively, the external terminal 400 generates support information for the user 500 on the basis of the analysis result and displays the support information on the display device or the like.

As described above, the biological information analysis device 1 according to the first embodiment performs the analysis of the plurality of biological information sets, such as heart rates of the user 500, measured over the plurality of time periods to calculate the average value of the plurality of biological information sets acquired at the mutually corresponding measurement times. Accordingly, even when a value of the biological information fluctuates from one of the time periods, such as “days”, to another, it is possible to obtain a typical value of the biological information of the user.

### Second Embodiment

Next, a description will be given of the second embodiment of the present invention. Note that, in the following description, the same components as those in the first embodiment described above are given the same reference numerals, and a description thereof is omitted.

In the first embodiment, the description has been given of the case where the averaging processing unit 110 calculates, for the biological information measured over the plurality of time periods, the average value at the mutually corresponding measurement times to obtain the typical value for the biological information of the user. By contrast, in the second embodiment, a biological information analysis device 1A further includes an abnormal value determination unit 16. The abnormal value determination unit 16 determines whether or not the measured biological information includes an abnormal value. The following will mainly describe components different from those in the first embodiment.

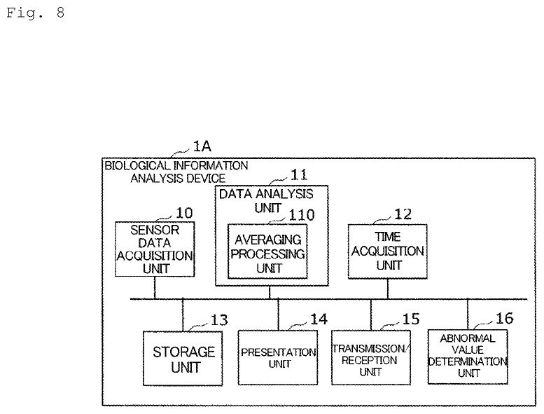

As illustrated in FIG. 8, the abnormal value determination unit 16 determines, on the basis of a standard set in advance, whether or not time-series data of the biological information includes an abnormal value. A result of the determination by the abnormal value determination unit 16 is input to the averaging processing unit 110. More specifically, the abnormal value determination unit 16 invalidates, on the basis of an upper-limit threshold and a lower-limit threshold each set in advance for a value of the biological information, measurement values of a value of the biological information over the upper-limit threshold and a value of the biological information under the lower-limit threshold. Values of the biological information from which the abnormal values are excluded by the abnormal value determination unit 16 are input to the averaging processing unit 110.

When it is determined by the abnormal value determination unit 16 that an abnormal value is included, the averaging processing unit 110 calculates, as a representative value, an average value of a plurality of biological information sets acquired at mutually corresponding measurement times in a plurality of time periods from which the abnormal value is excluded.

Operation Sequence of Biological Information Analysis System

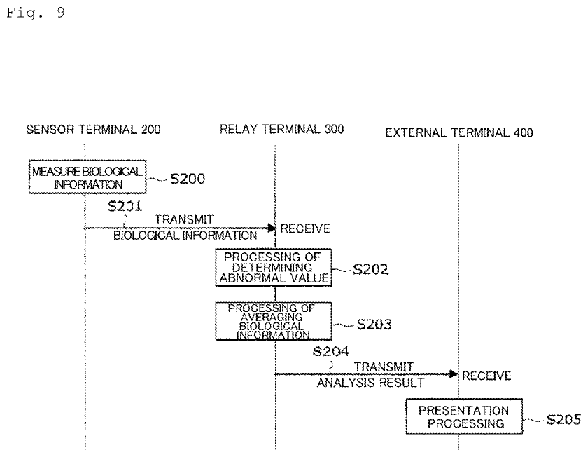

Next, referring to a sequence diagram illustrated in FIG. 9, a description will be given of an operation when the individual functions of the biological information analysis device 1A according to the present embodiment are implemented by a biological information analysis system including the sensor terminal 200, the relay terminal 300, and the external terminal 400 each illustrated in FIG. 6. Note that the individual functional blocks of the sensor terminal 200, the relay terminal 300, and the external terminal 400 are the same as those in the configuration illustrated in FIG. 6. It is assumed that the relay terminal 300 includes the abnormal value determination unit 16.

First, the sensor terminal 200 is worn by the user 500 to measure biological information of the user 500 over a plurality of time periods (Step S200). More specifically, the sensor terminal 200 measures a cardiac potential of the user 500 by using a heart rate measurement device (the sensor 201). The sensor data acquisition unit 202 acquires the cardiac potential from the sensor 201 to calculate a heart rate represented by digital data from the electrocardiographic waveform based on the cardiac potential. The acquired cardiac potential and the heart rate are stored in the data storage unit 203.

Then, the sensor terminal 200 transmits the measured biological information to the relay terminal 300 via the communication network NW (Step S201). More specifically, the data transmission unit 204 reads time-series data of the heart rate from the data storage unit 203 and transmits the time-series data to the relay terminal 300 via the communication network NW.

When receiving the time-series data of the biological information of the user 500 from the sensor terminal 200, the relay terminal 300 determines whether or not the received time-series data of the biological information includes the abnormal value in the abnormal value determination unit 16 (Step S202).

More specifically, the abnormal value determination unit 16 reads an upper-limit threshold (e.g., 190 bpm) and a lower-limit threshold (e.g., 40 bpm) of the heart rates stored in the data storage unit 302. The abnormal value determination unit 16 invalidates a value over the upper-limit threshold and a value under the lower-limit threshold in the received biological information of the user 500.

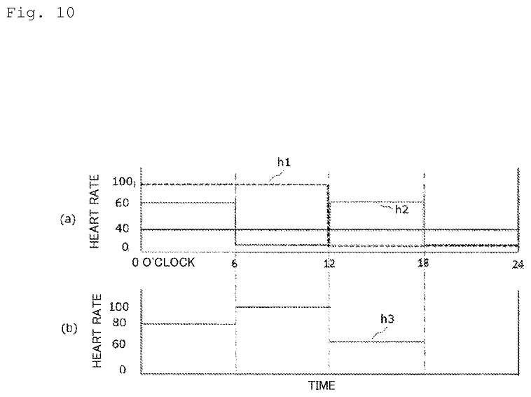

For example, as illustrated in (a) of FIG. 10, the heart rate h1 of the user 500 in a first time period is under the lower-limit threshold of 40 bpm in a time block from 12 o'clock to 24 o'clock. The heart rate h2 of the user in a second time period is similarly under the lower-limit threshold of 40 bpm in each of a time block from 6 o'clock to 12 o'clock and a time block from 18 o'clock to 24 o'clock. Therefore, the abnormal value determination unit 16 inputs, to the averaging processing unit 110, values of the heart rates except for the invalidated value of the heart rate under the lower-limit threshold.

Then, as illustrated in FIG. 9, the averaging processing unit 110 calculates, for the biological information of the user 500, an average value of a plurality of biological information sets acquired at mutually corresponding measurement times in a plurality of individual time periods on the basis of a result of the determination by the abnormal value determination unit 16 (Step S203). More specifically, the averaging processing unit 110 calculates, using Expression (1) described above, the average value h3 of the heart rates in the same time block in the first and second time periods from which the value determined to be the abnormal value is excluded, as illustrated in (b) of FIG. 10.

As illustrated in (b) of FIG. 10, an average value of the heart rates between 0 o'clock and 6 o'clock in each of the time periods is calculated to be 80 bpm, and an average value of the heart rates between 6 o'clock and 12 o'clock in each of the time periods is calculated to be 100 bpm due to the invalidated abnormal value. Likewise, an average value of the heart rates between 12 o'clock and 18 o'clock in each of the time periods is calculated to be 60 bpm. Between 18 o'clock and 24 o'clock, the heart rate in each of the first and second time periods is under the lower-limit threshold, and therefore the average value is not calculated. Specifically, in each of a time block from 6 o'clock to 12 o'clock and a time block from 12 o'clock to 18 o'clock, the number of data sets included in an average value At of the heart rates in Expression (1) described above is N=1, and is calculated as given by, e.g., At=(60)/1=60 bpm. In a time block from 18 o'clock to 24 o'clock, a result of calculating the average need not be returned on the assumption that data on the heart rates is absent (missing).

By thus specifying the abnormal value included in the biological information and removing the abnormal value from the calculation of the averaging processing, it is possible to reduce the influence of the abnormal valuate on the result of the analysis of the biological information. Therefore, it is possible to obtain a measurement value of the biological information corresponding to a behavior of the user 500 as a person under test irrespective of conditions for measurement by the sensor 201.

Back to FIG. 9, the relay terminal 300 transmits a result of analysis of the biological information obtained by performing the averaging processing to the external terminal 400 via the communication network NW (Step S204). Then, the external terminal 400 receives the analysis result. The external terminal 400 performs presentation processing on the basis of the analysis result (Step S205), displays the analysis result on a display device, generates support information for the user 500, and outputs the support information.

As described above, in the biological information analysis device 1A according to the second embodiment, even when the measured biological information of the user includes an abnormal value, the averaging processing is performed on the basis of the biological information from which the abnormal value is excluded, and the average value of the plurality of biological information sets acquired at the mutually corresponding measurement times in the plurality of individual time periods is calculated. Consequently, it is possible to reduce the influence of the abnormal value on the average value calculated as the typical value of the biological information and obtain more precise biological information corresponding to a habit of the user.

### Third Embodiment

Next, a description will be given of the third embodiment of the present invention. Note that, in the following description, the same components as those in the first and second embodiments described above are given the same reference numerals, and a description thereof is omitted.

In the second embodiment, the description has been given of the case where the biological information analysis device 1A includes the averaging processing unit 110 and the abnormal value determination unit 16, specifies the abnormal value in the biological information, and performs the averaging processing on the basis of a value of the biological information from which the abnormal value is excluded. By contrast, in a biological information analysis device 1B according to the third embodiment, a data analysis unit 11B includes a summarization unit 111, and calculates a statistical summary value based on an average value of biological information calculated by the averaging processing. The following will mainly describe components different from those in the first and second embodiments.

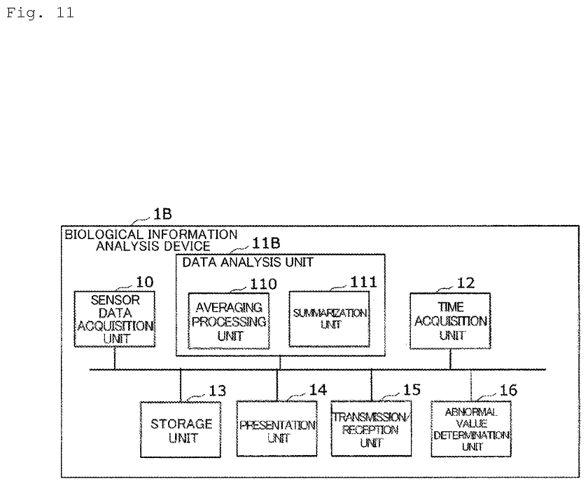

As illustrated in FIG. 11, the data analysis unit 11B of the biological information analysis device 1B includes the averaging processing unit 110 and the summarization unit 111.

The summarization unit 11 calculates, on the basis of time-series data of the average value of the biological information calculated by the averaging processing unit 110, the summary value, which is a value obtained by statistically summarizing the biological information during a randomly selected period. The summary value calculated by the summarization unit 111 includes at least one of a cumulative total value, an average value, a detailed breakdown representing a proportion accounted for by a duration period during which a randomly selected value of the biological information is continued in each of time periods in which the biological information is measured, a median value, a 25% level point, a 75% level point, a standard deviation, and a standard error.

Operation Sequence of Biological Information Analysis System

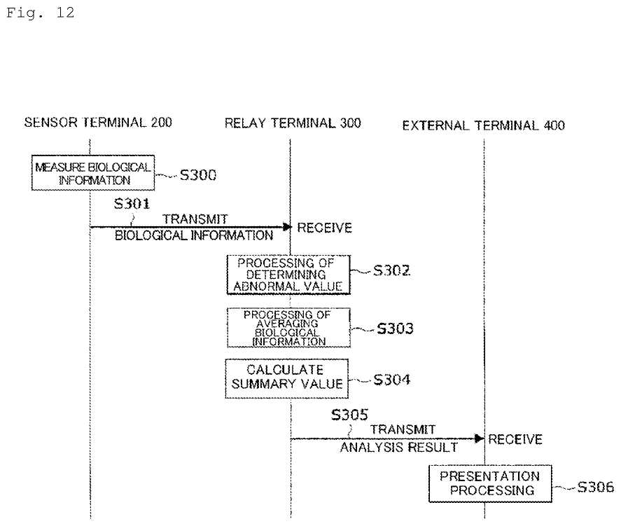

Next, using a case where individual functions of the biological information analysis device 1B according to the present embodiment are implemented by a biological information analysis system including the sensor terminal 200, the relay terminal 300, and the external terminal 400 each illustrated in FIG. 6 as an example, an operation will be described using a sequence diagram in FIG. 12. Note that, in the present embodiment, a description will be given of a case where the relay terminal 300 includes the data analysis unit 11B including the summarization unit 111 and the abnormal value determination unit 16.

First, the sensor terminal 200 is worn by the user 500 to measure biological information of the user 500 over a plurality of time periods (Step S300). More specifically, the sensor terminal 200 measures a cardiac potential of the user 500 by using a heart rate measurement device to calculate a heart rate represented by digital data from the electrocardiographic waveform based on the cardiac potential.

Next, the sensor terminal 200 transmits the biological information to the relay terminal 300 via the communication network NW (Step S301). When the relay terminal 300 receives time-series data of the biological information, the abnormal value determination unit 16 determines whether or not the received biological information includes an abnormal value (Step S302). When the abnormal value is specified by the abnormal value determination unit 16, the biological information from which the abnormal value is excluded is input to the averaging processing unit 110.

The averaging processing unit 110 calculates an average value of a plurality of biological information sets by using Expression (1) described above (Step S303). The averaging processing unit 110 delivers time-series data of the calculated average value of the biological information to the summarization unit 111.

Next, the summarization unit in calculates, on the basis of the time-series data of the average value of the biological information calculated by the averaging processing unit 110, the summary value representing the statistical summary of the biological information during a randomly selected period (Step S304). More specifically explaining the step using an example of time-series data of an average value of heart rates calculated by the averaging processing unit 110 which is illustrated in (b) of FIG. 10, the average value of the heart rates calculated by the summarization unit in on the assumption that the randomly selected period is six hours is calculated as (100+80+60)/3=80 bpm.

When k represents a sampling rate per hour, the summarization unit in calculates a cumulative total value of the heart rates as (100+80+60) [bpm]×6 [hours]×k [points].

As the detailed breakdown representing the proportion accounted for by the total duration period of the randomly selected value of the biological information in each of the time periods in which the biological information is measured, the summarization unit 111 can also determine a proportion accounted for by a total duration period of each of average values of the heart rates in one day such that “periods during which the heart rate was 100 bpm, 80 bpm, and 60 bpm each account for 33%”, as illustrated in (b) of FIG. 10.

Then, the relay terminal 300 transmits, as an analysis result, the summary value of the biological information determined by the summarization unit 111 to the external terminal 400 via the communication network NW (Step S305). Then, when receiving the analysis result, the external terminal 400 performs the processing of presenting the analysis result (Step S306). More specifically, the external terminal 400 may also display the summary value of the biological information received as the analysis result on a display device included in the external terminal 400 or generate information supporting the user 500 on the basis of the summary value and display the support information on the display device.

As described above, in the biological information analysis device 1B according to the third embodiment, the summarization unit 111 calculates, on the basis of the time-series data of the average value of the biological information of the user, the summary value representing the statistical summary during the randomly selected period. Therefore, it is possible to statistically obtain the biological information of the user in one time period as a summary.

### Fourth Embodiment

Next, a description will be given of the fourth embodiment of the present invention. Note that, in the following description, the same components as those in the first to third embodiments described above are given the same reference numerals, and a description thereof is omitted.

In the first to third embodiments, the description has been given using the heart rate, which is a quantitative variable, as a specific example of the biological information. By contrast, in the fourth embodiment, a description will be given of a case where biological information to be analyzed is a qualitative variable which is not permitted to have a middle value, such as a posture of the user. A data analysis unit 11C included in a biological information analysis device 1C according to the fourth embodiment includes a summarization unit 111C that analyzes the biological information (hereinafter referred to as “biological state information”) representing the qualitative variable. The following will mainly describe components different from those in the first to third embodiments.

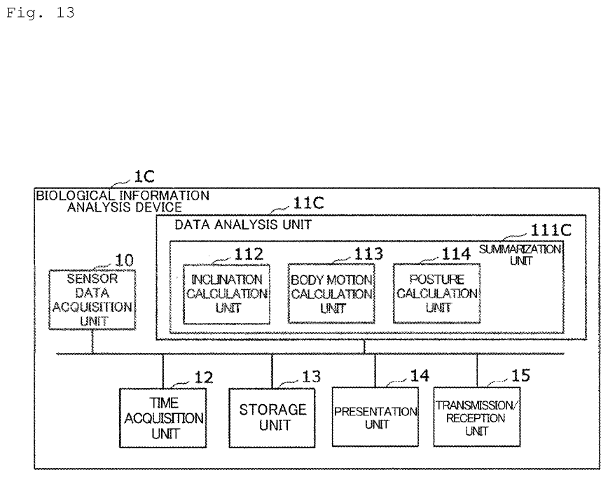

As illustrated in FIG. 13, the biological information analysis device 1C includes a data analysis unit 11C. The data analysis unit 11C includes the summarization unit 111C.

The summarization unit 111C calculates the biological state information, which is the qualitative variable, from time-series data of the biological information acquired by the sensor data acquisition unit 10, and calculates a summary value by statistically summarizing the biological state information on the basis of frequencies of occurrence of a value of the biological state information at mutually corresponding measurement times in a plurality of individual time periods.

In the present embodiment, the biological state information to be analyzed by the data analysis unit 11C is the qualitative variable. When it is assumed that a state in which the user is recumbent (lying flat) is represented by “o” and a state where the user is sitting or standing such as in a standing position is represented by “i”, the biological state information is a variable having a property of being categorized into either of the two values. Since the biological information such as the heart rate used as an object to be subjected to data analysis in the first to third embodiments is a so-called quantitative variable, it is possible to calculate an average value represented as an intermediate value in the time-series data. However, in the biological state information to be processed in the present embodiment, an intermediate value, e.g., a value of “0.5” makes no sense in the example of the posture of the user described above.

In the present embodiment, the summarization unit 111C assumes that, when biological state information sets acquired at mutually corresponding measurement times in a plurality of time periods (plurality of days) have the same value, an average value is the same value. Meanwhile, when the biological state information sets measured at the mutually corresponding measurement times in the plurality of days have different values, the summarization unit 111C divides a measurement period on the basis of a ratio among the individual values.

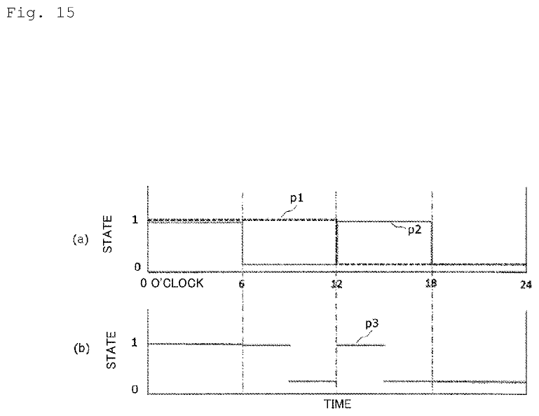

More specifically, as illustrated in (a) of FIG. 15, the biological state information on a first day (in a first time period) is such that a state p1 has “1” representing a sitting or standing state from 0 o'clock to 12 o'clock and has “o” representing a recumbent state from 12 o'clock to 24 o'clock. In addition, the biological state information on a second day (in a second time period) is such that a state p2 has “1” representing the sitting or standing state in time blocks from 0 o'clock to 6 o'clock and from 12 o'clock to 18 o'clock and has “o” representing the recumbent state in time blocks from 6 o'clock to 12 o'clock and from 18 o'clock to 24 o'clock.

(b) of FIG. 15 illustrates a state p3 representing an average of the biological state information over two days (in two periods) in (a) of FIG. 15 that has been calculated by the summarization unit 111C. As illustrated in (b) of FIG. 15, the state p3 represented by the biological state information from 0 o'clock to 6 o'clock and from 18 o'clock to 24 o'clock in one day (one period) has the same value on each of the first day and the second day. Accordingly, the same value is calculated for each of the first day and the second day.

Meanwhile, in the time blocks from 6 o'clock to 12 o'clock and from 12 o'clock to 18 o'clock, a value on the first day is different from a value on the second day. Accordingly, each of a period from 6 o'clock to 12 o'clock and a period from 12 o'clock to 18 o'clock is halved, and “1” is allocated as the state p3 to a time block from 6 o'clock to 9 o'clock, while “o” is allocated as the state p3 to a time block from 9 o'clock to 12 o'clock.

Likewise, the time block from 12 o'clock to 18 o'clock is also halved, and “1” and “o” are combined with each other to be allocated as the state p3.

The summarization unit 111C determines an average of the values representing the biological state information through time division as described above. The summarization unit 111C also calculates, on the basis of the frequency of the value representing the average of the biological state information determined through the time division, a summary value, which is a value by statistically summarizing the biological state information.

More specifically, in the example in (b) of FIG. 15, the summarization unit 111C calculates a summary such that “a total duration period during which the state takes ‘1’ and a total duration period during which the state takes ‘0’ each account for 50% of one day (1 period)”. Note that the number of values representing states is not limited to 2. The same calculation is possible even when the number of the values representing the states is 3 or more. For example, when a state have three values of “0, 1, and 2”, and a combination of “0, 0, and 1” exists for 30 minutes, it is appropriate to calculate a summary such that “a total duration period during which the state takes “0” is 20 minutes and a total duration period during which the state takes “1” is 10 minutes”.

In the present embodiment, the summarization unit 111C includes an inclination calculation unit 112, a body motion calculation unit 113, and a posture calculation unit 114.

The inclination calculation unit 112 calculates, from 3-axis acceleration data, an inclination of the sensor 106 worn by the user.

The body motion calculation unit 113 calculates, from the 3-axis acceleration data, a magnitude of a body motion of the user.

The posture calculation unit 114 calculates, from the inclination calculated by the inclination calculation unit 112, the posture of the user. The value calculated by the posture calculation unit 114 is used by the summarization unit 111C as a value representing the biological state information of the user.

The inclination calculation unit 112 calculates, from the 3-axis acceleration data obtained by the sensor data acquisition unit 10, θ and φ as the inclination of the sensor 106 with respect to a gravity acceleration on the basis of the following expressions. Note that the inclination calculation unit 112 uses the 3-axis acceleration data sampled by the sensor data acquisition unit 10 at a sampling rate of, e.g., 25 Hz.

Meanwhile, θ (−90≤θ<270) represents an inclination of a Z-axis of the acceleration sensor with respect to a vertical direction, φ (−90≤φ<270) represents an inclination of an X-axis of the acceleration sensor with respect to the vertical direction, and a unit is degree.

$$\begin{matrix}Formula 2 & \\ θ = \frac{180}{π} {cos}^{- 1} ( \frac{{A}_{z}}{\sqrt{{A}_{x}^{2} + {A}_{y}^{2} + {A}_{z}^{2}}} ) + 90 ( {A}_{y} \geq 0 ) \\ θ = - \frac{180}{π} {cos}^{- 1} ( \frac{{A}_{z}}{\sqrt{{A}_{x}^{2} + {A}_{y}^{2} + {A}_{z}^{2}}} ) + 90 ( {A}_{y} < 0 ) & ( 2 ) \\ Formula 3 & \\ ∅ = \frac{180}{π} {cos}^{- 1} ( \frac{{A}_{z}}{\sqrt{{A}_{x}^{2} + {A}_{y}^{2} + {A}_{z}^{2}}} ) + 90 ( {A}_{y} \geq 0 ) \\ ∅ = - \frac{180}{π} {cos}^{- 1} ( \frac{{A}_{z}}{\sqrt{{A}_{x}^{2} + {A}_{y}^{2} + {A}_{z}^{2}}} ) + 90 ( {A}_{y} < 0 ) & ( 3 )\end{matrix}$$

Each of Ax, Ay, and Az represents an output value from the acceleration sensor, and uses a gravity acceleration G (1.0 G≈9.8 m/s²) as a unit. In each of Expressions (2) and (3), a ratio of a single-axis measurement value to a magnitude (norm) of a resultant vector of the output values from the acceleration sensor is determined, and an inverse function of a cosine of angle (cosine) is further determined to calculate the inclination as a value having an angle dimension.

In Ax, Ay, and Az in Expressions (2) and (3), the output values from the acceleration sensor may be substituted directly or, instead, values obtained by appropriately using a low pass filter (e.g., a FIR filter or moving average filter) for smoothing may also be used.

The posture calculation unit 114 compares values of 6 and p calculated on the basis of Expressions (2) and (3) to respective thresholds to calculate the posture. For example, an inclination of the sensor terminal 200 including an acceleration sensor as illustrated in FIG. 6 reflects an inclination of an upper body of the user 500 wearing the sensor terminal 200. Accordingly, from the inclination of the sensor terminal 200, the posture of the user 500 can be calculated. For example, the posture is categorized and calculated in accordance with the following calculation method.

(i) Standing Position (Upright Standing): when 30≤θ<140 is satisfied.

(ii) Standing Position (Inverted Standing): when 6<−40 or 220<θ is satisfied.

(iii) Recumbent Position (Left-Side-Up Position): when (φ≤−50 or 230<φ) and (−40≤θ<30) are satisfied or when (φ≤−50 or 230<φ) and (140≤θ<220) are satisfied.

(iv) Recumbent Position (Right-Side-Up Position): when (50<φ<130) and (−40≤θ<30) are satisfied or when (50<φ<130) and (140≤θ<220) are satisfied.

(v) Recumbent Position (Face-Up Position): when (130≤φ≤230) and (−40≤θ<30) are satisfied or when (130≤φ≤230) and (140<θ<220) are satisfied.

(vi) Recumbent Position (Face-Down Position): when (−50≤φ≤50) and (−40≤θ<30) are satisfied or when (−50≤φ≤50) and (140<θ<220) are satisfied.

Definitions of (i) to (vi) in the calculation described above may be set (stored) appropriately as a table of θ and φ as illustrated below in Table 1 in the posture calculation unit 114.

TABLE 1

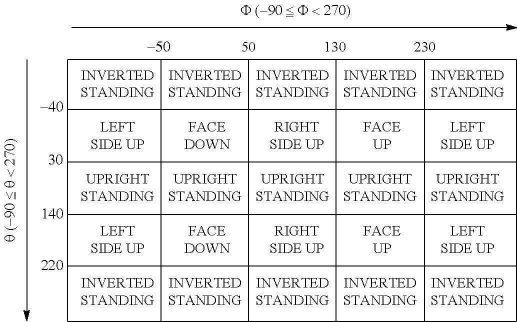

When it is assumed that “0” represents a recumbent position and “1” represents a standing position on the basis of the posture of the user calculated by the posture calculation unit 114, a qualitative variable is provided.

Operation Sequence of Biological Information Analysis System

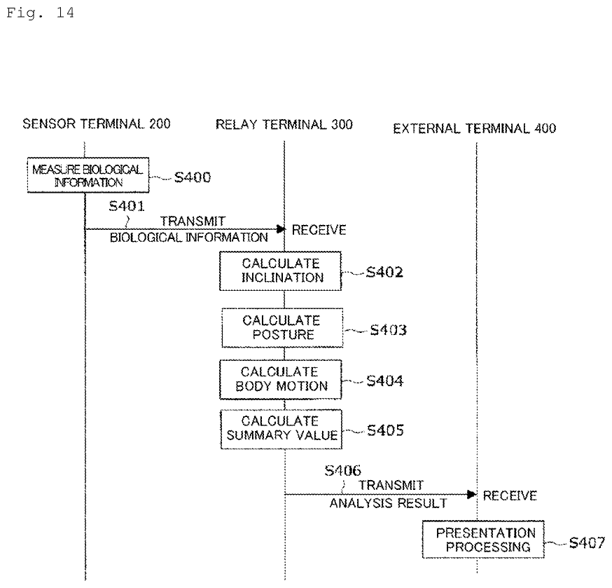

Next, using a sequence diagram in FIG. 14, a description will be given of an operation of a biological information analysis system in which individual functions of the biological information analysis device 1C according to the present embodiment are implemented by the sensor terminal 200, the relay terminal 300, and the external terminal 400 each illustrated in FIG. 6. Note that, in the present embodiment, the description will be given of a case where the relay terminal 300 has the data analysis unit 11C including the summarization unit 111C.

First, the sensor terminal 200 including a 3-axis acceleration sensor is worn by the user 500, and 3-axis accelerations of the user 500 are measured over a plurality of time periods (Step S400). The sensor terminal 200 removes noise from measured acceleration data or obtains acceleration data resulting from sampling at a sampling rate of, e.g., 25 Hz.

Next, the sensor terminal 200 transmits the acceleration data to the relay terminal 300 via the communication network NW (Step S401). When the relay terminal 300 receives time-series data of the accelerations, the inclination calculation unit 112 calculates an inclination from the received 3-axis acceleration data by using Expressions (2) and (3) described above (Step S402).

Next, the posture calculation unit 114 calculates the posture of the user 500 on the basis of the inclination calculated in Step S402 (Step S403). More specifically, the posture calculation unit 114 determines the posture as the biological state information of the user 500 with reference to the table illustrated in Table 1 described above. The value calculated by the posture calculation unit 114 is used as a value representing the biological state information of the user 500 by the summarization unit 111C. Note that, as illustrated in (a) of FIG. 15, the biological state information obtained by the posture calculation unit 114 is represented by two values such that “0” represents the recumbent position and “1” represents the standing position as in the state p1 of the user 500 on the first day (in the first time period) and the state p2 of the user 500 on the second day (in the second time period).

Then, the body motion calculation unit 113 calculates a magnitude of a body motion of the user 500 from the 3-axis acceleration data (Step S404). Subsequently, the summarization unit 111C calculates a statistical summary value of the biological state information during a randomly selected period on the basis of time-series data ((a) of FIG. 15) of the posture serving as the biological state information of the user 500 determined by the posture calculation unit 114 (Steps S405).

More specifically, as illustrated in (b) of FIG. 15, the summarization unit 111C determines an average of values representing the biological state information through time division. In addition, the summarization unit 111C calculates a summary value by statistically summarizing the biological state information on the basis of a frequency of a value representing the average of the biological state information determined through the time division.

Then, the relay terminal 300 transmits the calculated summary value as an analysis result to the external terminal 400 via the communication network NW (Step S406). Then, when receiving the analysis result, the external terminal 400 performs processing of presenting the analysis result (Step S407). More specifically, the external terminal 400 may display the summary value of the biological state information received as the analysis result on the display device included in the external terminal 400 or may also generate information supporting the user 500 on the basis of the summary value and display the support information on the display device.

As described above, in the biological information analysis device 1C according to the fourth embodiment, the summarization unit 111C calculates, on the basis of the time-series data of the biological state information related to the qualitative variable such as the posture of the user, the summary value representing the statistical summary during the randomly selected period. Accordingly, even when the biological state information is the qualitative variable, it is possible to statistically obtain the biological state information in one day (one time period) of the user as a summary.

While the description has been given heretofore of the embodiments of the biological information analysis device, the biological information analysis method, and the biological information analysis system each according to the present invention, the present invention is not limited to the described embodiments. Various modifications that can be easily conceived of by a person skilled in the art can be made in the present invention within the scope of the appended claims.

In the embodiments described above, the description has been given of the case where the biological information to be measured and calculated by the sensors 106 and 201 is the cardiac potential or the acceleration. However, the biological information is not limited thereto, and may also be, e.g., a myopotential, a heart rate, a pulse beat, a blood pressure, breathing, a posture, walking, a moving speed, a position, an operation, an exercise intensity, a body motion, an amount of activity, or the like.

In the embodiments described above, the description has been given of the case where, in the specific example, the relay terminal 300 includes the data analysis unit 11. However, the individual functions of the data analysis unit 11 may also be distributed to and implemented by the sensor terminal 200, the relay terminal 300, and the external terminal 400.

For example, it may also be possible that the sensor terminal 200 having a first data analysis unit, the relay terminal 300 having a second data analysis unit, and the external terminal 400 having a third data analysis unit cooperate to analyze time-series data of biological information over a plurality of time periods and calculate, from a plurality of biological information sets acquired at mutually corresponding measurement times in the plurality of individual time periods, a representative value of the plurality of biological information sets.

### REFERENCE SIGNS LIST

1, 1A, 1B, 1C Biological information analysis device
10, 202 Sensor data acquisition unit
11, 304 Data analysis unit
12, 303 Time acquisition unit
13 Storage unit
14, 404 Presentation unit
15 Transmission/reception unit
101 Bus
102 Arithmetic device
103 CPU
104 Main storage device
105 Communication Interface
106,201 Sensor
107 External storage device
108 Clock
109 Display device
200 Sensor terminal
300 Relay terminal
400 External terminal
203, 302, 402 Data storage unit
204, 305 Data transmission unit
301, 401 Data reception unit
403 Presentation processing unit

1.-8. (canceled)

9. A biological information analysis device comprising:
a sensor data acquisition circuit configured to acquire biological information measured by a sensor; and
a data analyzer configured to:
analyze time-series data of the biological information over a plurality of time periods; and
calculate, from a plurality of biological information sets acquired at mutually corresponding measurement times in the plurality of time periods, a representative value of the plurality of biological information sets.

10. The biological information analysis device according to claim 9, further comprising an abnormal value determination circuit configured to:
determine, based on a preset reference, whether the plurality of biological information sets includes an abnormal value, wherein the data analyzer is configured to in response to a determination that the abnormal value is included in the plurality of biological information sets, exclude the abnormal value from the plurality of biological information sets when calculating the representative value of the plurality of biological information sets.

11. The biological information analysis device according to claim 9, wherein the data analyzer includes an averaging processor configured to calculate, as the representative value, an average value of the plurality of biological information sets.

12. The biological information analysis device according to claim 11, wherein the data analyzer further includes a summarization device configured to calculate, based on time-series data of the average value of the plurality of biological information sets, a summary value, wherein the summary value is obtained by statistically summarizing the plurality of biological information sets for each of randomly selected periods included in the plurality of time periods, and wherein the summary value includes a cumulative total value, an average value, a detailed breakdown representing a proportion accounted for by a total duration period of a randomly selected value of the biological information in each of the plurality of time periods, a median value, a 25% level point, a 75% level point, a standard deviation, or a standard error.

13. The biological information analysis device according to claim 12, wherein the summarization device is further configured to:
calculate biological state information based on the time-series data of the biological information over the plurality of time periods, wherein the biological state information is a qualitative variable; and
calculates the summary value by statistically summarizing the biological state information based on frequencies of a value of the biological state information at mutually corresponding measurement times in the plurality of time periods.

14. A biological information analysis method comprising:
acquiring biological information measured by a sensor; and
analyzing time-series data of the biological information over a plurality of time periods to calculate, from a plurality of biological information sets acquired at mutually corresponding measurement times in the plurality of time periods, a representative value of the plurality of biological information sets.

15. The biological information analysis method according to claim 14, further comprising:
determining, based on a preset reference, whether the plurality of biological information sets includes an abnormal value; and
in response to a determining that the abnormal value is included in the plurality of biological information sets, excluding the abnormal value from the plurality of biological information sets when calculating the representative value of the plurality of biological information sets.

16. The biological information analysis method according to claim 14, calculating the representative value comprises calculating, as the representative value, an average value of the plurality of biological information sets.

17. The biological information analysis method according to claim 16, further comprising:
calculating, based on time-series data of the average value of the plurality of biological information sets, a summary value, wherein the summary value is obtained by statistically summarizing the plurality of biological information sets for each of randomly selected periods included in the plurality of time periods, and wherein the summary value includes a cumulative total value, an average value, a detailed breakdown representing a proportion accounted for by a total duration period of a randomly selected value of the biological information in each of the plurality of time periods, a median value, a 25% level point, a 75% level point, a standard deviation, or a standard error.

18. The biological information analysis method according to claim 17, further comprising:
calculating biological state information based on the time-series data of the biological information over the plurality of time periods, wherein the biological state information is a qualitative variable, wherein calculating the summary value comprises calculating the summary value by statistically summarizing the biological state information based on frequencies of a value of the biological state information at mutually corresponding measurement times in the plurality of time periods.

19. A biological information analysis system comprising:
a sensor terminal configured to output biological information measured by a sensor worn by a user;
a relay terminal configured to receive the biological information from the sensor terminal and output the biological information; and
an external terminal configured to receive the biological information from the sensor terminal or the relay terminal and causes a display device to display the biological information, wherein the sensor terminal, the relay terminal, or the external terminal includes:
a sensor data acquisition device configured to acquire the biological information;
a data analyzer configured to analyze time-series data of the biological information over a plurality of time periods to calculate, from a plurality of biological information sets acquired at mutually corresponding measurement times in the plurality of time periods, a representative value of the plurality of biological information sets; and
a presentation device configured to output the representative value of the plurality of biological information sets as a portion of the biological information.
